(19) United States
(12) Patent Application Publication
(10) Pub. No.: US 2018/0099093 A1
(43) Pub. Date: Apr. 12, 2018

(54) COMPARTMENTALIZED AUTO-INJECTION SYSTEM

(71) Applicant: AdrenaCard, Inc., Minneapolis, MN (US)

(72) Inventors: Tyler Ebert, Weston, WI (US); Christopher Kuehn, Shoreview, MN (US)

(21) Appl. No.: 15/564,072

(22) PCT Filed: Mar. 16, 2016
(86) PCT No.: PCT/US16/22608

§ 371 Date: Oct. 3, 2017

Related U.S. Application Data
(60) Provisional application No. 62/142,508, filed on Apr. 3, 2015.

(51) Int. Cl. A61M 5/20; A61M 5/32

(52) U.S. Cl. CPC A61M 5/2033; A61M 5/32; A61M 2005/314; A61M 2005/208; A61M 2005/206

(57) ABSTRACT

A needle injection system (“NIS”) comprised of a unique compartmental arrangement. More specifically, an NIS that utilizes two separate energy storage units, wherein one energy storage unit drives the needle from a first position inside a housing to a second position outside of the housing, and the second energy storage unit applies pressure to the medicament storage container in order expel the medication through the needle. A fluid communication channel is located between the medicament storage container and needle. The fluid communication channel contains a seal that prevents fluid flow to, and fluid communication with, the needle until the device is intentionally activated. The seal can be a geometrical seal or a frangible seal capable of being punctured. The geometrical seal can be comprised of a system of O-rings and a sliding alignment hole, which, when aligned, completes the fluid communication. The frangible seal can face perpendicular to the needle.

### CROSS-REFERENCE TO RELATED APPLICATION

This application claims the benefit of U.S. Provisional Patent Application No. 62/142,508, filed on Apr. 3, 2015, titled COMPARTMENTALIZED AUTOMATIC INJECTOR.

### FIELD OF THE DISCLOSURE

The disclosed invention generally relates to a Needle-Injection System. More specifically, the disclosed system relates to an automatic injector having two separate energy storage members that act in unison on a needle and a medicament storage container to expel the entirety of the injector's contents upon activation.

### BACKGROUND OF THE INVENTION

An automatic Needle-Injection System (hereinafter “NIS”), or “auto-injector,” is a device designed for the self-administration of a pre-measured dose of medication, very often in an emergency situation. A multitude of medications can be administered through the use of an auto-injector, of which the intent is intra-muscular drug delivery. Examples of medications administered include anti-opiates, anti-toxins, anti-venoms, emergency glucose medication for diabetics, and emergency doses of epinephrine as a treatment for acute anaphylaxis.

A typical auto-injector contains one of two types of injection systems, a cartridge injection system or a syringe injection system. In a cartridge injection system, the needle component is usually manufactured separately from the medicament storage container as a glass or plastic syringe, and, upon activation of the auto-injector, the needle punctures a seal in the medicament storage container. Thereafter, the needle is in open fluid communication with the medication and the dose of medication is allowed to freely flow through the needle component.

In a syringe injection system, the needle is built into the medicament storage container and is in constant open fluid communication with the medicine, similar to a non-NIS hand-activated syringe. When pressure is applied to the medicament storage container, usually by a plunger contained within the housing of the medicament storage container, the pressure on the fluid medication causes the dosage to flow out through the needle. While these two systems are fairly common, dosage delivery mechanisms are often inaccurate.

The activation mechanisms of auto-injectors vary widely, but a standard injection procedure contains three to four steps. The first step is to remove a device's outer protective casing, which assists in keeping the device inside its recommended mechanical or thermal storage limits. The second step is to remove a safety unit, such as a pin or a tab. The safety unit protects the user from accidental activation of the device. The third step is to position the injector on the correct injection site. The fourth, and final, step is to activate the device either by placing pressure on the proximal (i.e., needle) end of the device or by using a separate activation button or lever positioned elsewhere on the device. However, even though a fairly standard activation procedure exists for auto-injectors, the design of the activation process of various auto-injectors currently on the market confuses users and is not intuitive.

Despite some of the standardization in the auto-injector industry, many devices are still fairly bulky and do not address user needs. More specifically, individuals that require the use of auto-injectors usually require them in emergencies. Therefore, the auto-injector needs to be easily accessible at almost all times of the day. Because of the bulky size of current auto-injectors, individuals are more likely store them in bags or purses and the auto-injector can fall to the bottom of the bag or purse and be difficult to find or access in an emergency. Therefore, an auto-injector is needed that provides an accurate dose, has an intuitive activation process, and is designed to have a more accessible design that makes it easy for individuals to find in emergency situations.

### SUMMARY OF THE INVENTION

The depicted invention pertains to an auto-injector that expels the entirety of its contents upon activation and that provides an accurate dosage delivery mechanism, an intuitive, user-friendly activation process, and a compact design. The invention is an NIS that contains a unique compartmental arrangement, which allows the NIS to be built into a smaller, relatively flat container. This particular arrangement utilizes two separate energy storage units. One energy storage unit drives the needle from a first position inside a housing to a second position outside of the housing, and the second energy storage unit applies pressure to the medicament storage container in order to pressurize the medication and expel it through the needle. This particular design contains a fluid communication channel located between the medicament storage container and needle. The fluid communication channel contains a seal that prevents fluid flow to the needle until the device is intentionally activated. The seal can be a geometrical seal or a frangible seal that is capable of being punctured. The geometrical seal can be comprised of a system of O-rings and a sliding alignment hole, which, when aligned, completes the fluid communication. The frangible seal can operate much like a standard cartridge system.

### BRIEF DESCRIPTION OF THE DRAWINGS

FIG. 1 is a perspective view of the compartmentalized auto-injection system in one embodiment of the current disclosure.

FIG. 2 is a top view of the internals of the compartmentalized auto-injection system in one embodiment of the current disclosure.

FIG. 3A illustrates one embodiment of the main delivery mechanisms of the compartmentalized auto-injection system in a pre-activation state.

FIG. 3B illustrates one embodiment of the main delivery mechanisms of the compartmentalized auto-injection system in a post-activation state.

FIG. 4A illustrates one embodiment of the main delivery mechanisms of the compartmentalized auto-injection system in a pre-activation state.

FIG. 4B illustrates one embodiment of the main delivery mechanisms of the compartmentalized auto-injection system in a post-activation state.

FIG. 5 is a perspective cross-sectional view of the needle housing component in one embodiment of the current disclosure.

FIG. 6 is a perspective, cross-sectional view of the fluid communication manifold in one embodiment of the current disclosure.

FIG. 7 is a top view of the internals of the compartmentalized auto-injection system in one embodiment of the current disclosure.

FIG. 8A illustrates one embodiment of the main delivery mechanisms of the compartmentalized auto-injection system in a pre-activation state.

FIG. 8B illustrates one embodiment of the main delivery mechanisms of the compartmentalized auto-injection system in a post-activation state.

FIG. 9 is a perspective, cross-sectional view of the injection components of the compartmentalized auto-injection system in one embodiment of the current disclosure.

FIG. 10 is a perspective, cross-sectional view of the fluid communication manifold in one embodiment of the current disclosure.

FIG. 11 is a top view of the needle housing component in one embodiment of the current disclosure.

FIG. 12 is a detailed description of one method by which the compartmentalized auto-injection system can be filled.

FIG. 13 is a detailed description of one method by which the compartmentalized auto-injection system can be filled.

FIG. 14A illustrates one embodiment of the main delivery mechanisms of the compartmentalized auto-injection system in a pre-activation state.

FIG. 14B illustrates one embodiment of the main delivery mechanisms of the compartmentalized auto-injection system in a post-activation state.

FIG. 15 illustrates one embodiment of the main delivery mechanisms of the compartmentalized auto-injection system in a pre-activation state.

FIG. 16 is a perspective view of the compartmentalized auto-injection system in a protective external case in one embodiment of the current disclosure.

FIG. 17A is a perspective view of the compartmentalized auto-injection system and a protective external case in one embodiment of the current disclosure.

FIG. 17B is a perspective view of the compartmentalized auto-injection system and a protective external case in one embodiment of the current disclosure.

FIG. 18 is a perspective view of the compartmentalized auto-injection system in a protective external case in one embodiment of the current disclosure.

FIG. 19 is a perspective view of the compartmentalized auto-injection system in one embodiment of the current disclosure.

FIG. 20A is a perspective view of the compartmentalized auto-injection system in one embodiment of the current disclosure.

FIG. 20B is a perspective view of the compartmentalized auto-injection system in one embodiment of the current disclosure.

FIG. 21 is a perspective view of the compartmentalized auto-injection system and a protective external case in one embodiment of the current disclosure.

FIG. 22 is a perspective view of the compartmentalized auto-injection system and a protective external case outfitted with a belt-clip in one embodiment of the current disclosure.

FIG. 23 is a perspective view of the compartmentalized auto-injection system and a protective external case adhered to an electronic device in one embodiment of the current disclosure.

FIG. 24 is a perspective view of a protective wallet designed to store the compartmentalized auto-injection system.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims. It is understood that various omissions and substitutions of equivalents are contemplated as circumstances may suggest or render expedient, but these are intended to cover applications or embodiments without departing from the spirit or scope of the claims attached hereto. Also, it is to be understood that the phraseology and terminology used herein are for the purpose of description and should not be regarded as limiting.

The disclosed invention is an auto injector in which the injection system is designed to be substantially small and thin in one axis, yet still easily gripped during the injection process. The overall device is stored in an external case 1601 meant to assist the device in staying within its thermal or mechanical storage limits. The external case 1601 can be constructed out of a wide variety of materials, including, but not limited to, injection-molded thermoplastics, cast or machined metallic material, or stamped and rolled sheet metal. In one embodiment, the external case 1601 contains a thermally insulating material, such as an adhesive-backed aluminum tape or a foam disposed between the inside surface of the external case 1601 and the outer faces of the auto-injector, with the intent of protecting the device from adverse thermal elements. In a preferred embodiment, the auto-injector has a cartridge that is manufacturer-filled with a medication. However, in some embodiments, the cartridge can be user-filled with the medication.

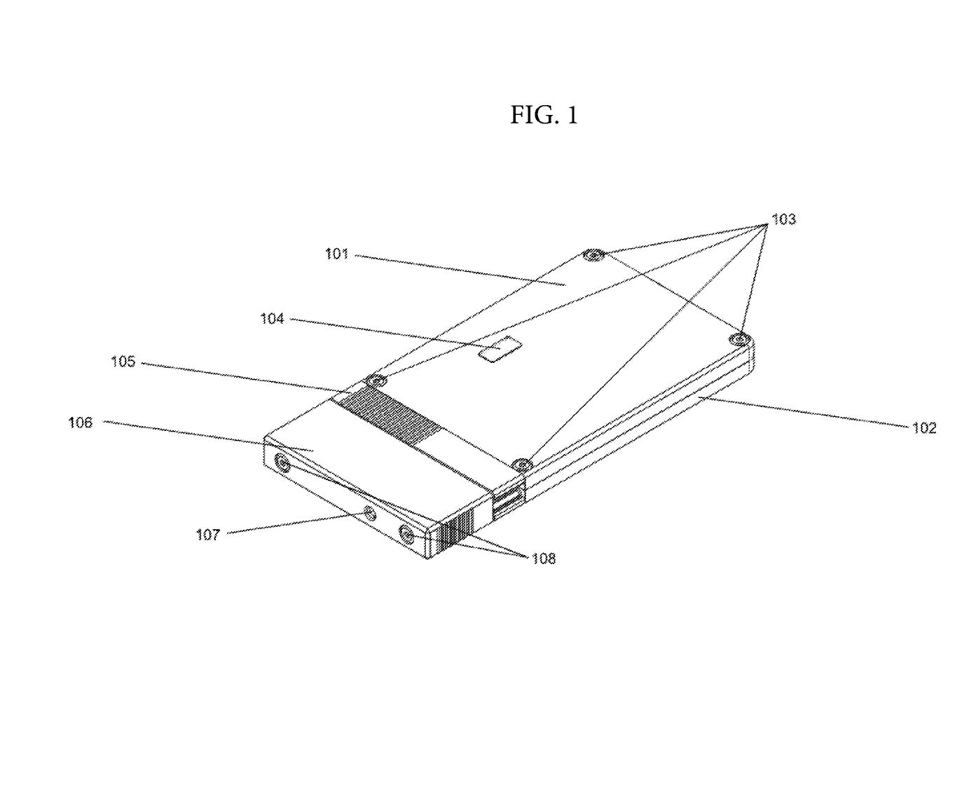

Externally, the auto-injector can have a thin geometrical form factor that is defined by a housing. The housing, as illustrated in FIG. 1, can be comprised of the outer faces of a top housing component 101 and a bottom housing component 102 that contain the device's injection components. The top and bottom housing components 101, 102 may be machined or cast metallic material such as aluminum, magnesium, or zinc. Alternatively, they may be injection molded out of a thermoplastic material. The main functionality of the top and bottom housing components 101, 102 is to provide structural stability for the injection components held within as well as to provide very precise anchoring positions for the moving injector components held within. The top and bottom housing components 101, 102 are joined by a system of small fasteners 103 positioned in the corners of the housing components 101, 102. The top housing component 101 can contain four counter-bore holes that allow for the passage of the fastener through the top housing component 101, while the bottom housing component 102 can contain a threaded hole to facilitate joining of the top and bottom housing components 101, 102.

Both the top and bottom housing components 101, 102 can contain a viewing port 104 constructed of a clear acrylic or glass material that allows the user to see through the top and bottom housing components 101, 102. The top and bottom housing components 101, 102 can also contain a clear syringe 212 disposed inside of the housing that contains medication. The viewing port 104 enables a user to view the medication held within the syringe 212 of the auto-injector in order to determine if any discoloration or precipitates have appeared in the medication.

In some embodiments, a safety tab 105 is located towards the proximal end of the device, between the needle triggering mechanism component 106 and a sunken ridge in the top and bottom housing components 101, 102. The safety tab 105 provides a way of safely allowing the auto-injector to be carried and removed from its external case 1601 without worry of accidental device activation. The safety tab 105 accomplishes this by blocking linear movement of the triggering mechanism component 106 from the proximal end of the auto-injector, which contains the needle port hole 107, towards the distal end. Because movement of the triggering mechanism component 106 would normally cause activation of the auto-injector, the safety tab 105 acts as a physical barrier to prevent activation. As the second step in the activation process after removal of the external case 1601, a user can remove the safety tab 105 by pulling on it laterally. This movement displaces the safety tab 105 from the side of the housing to allow for movement of the triggering mechanism component 106.

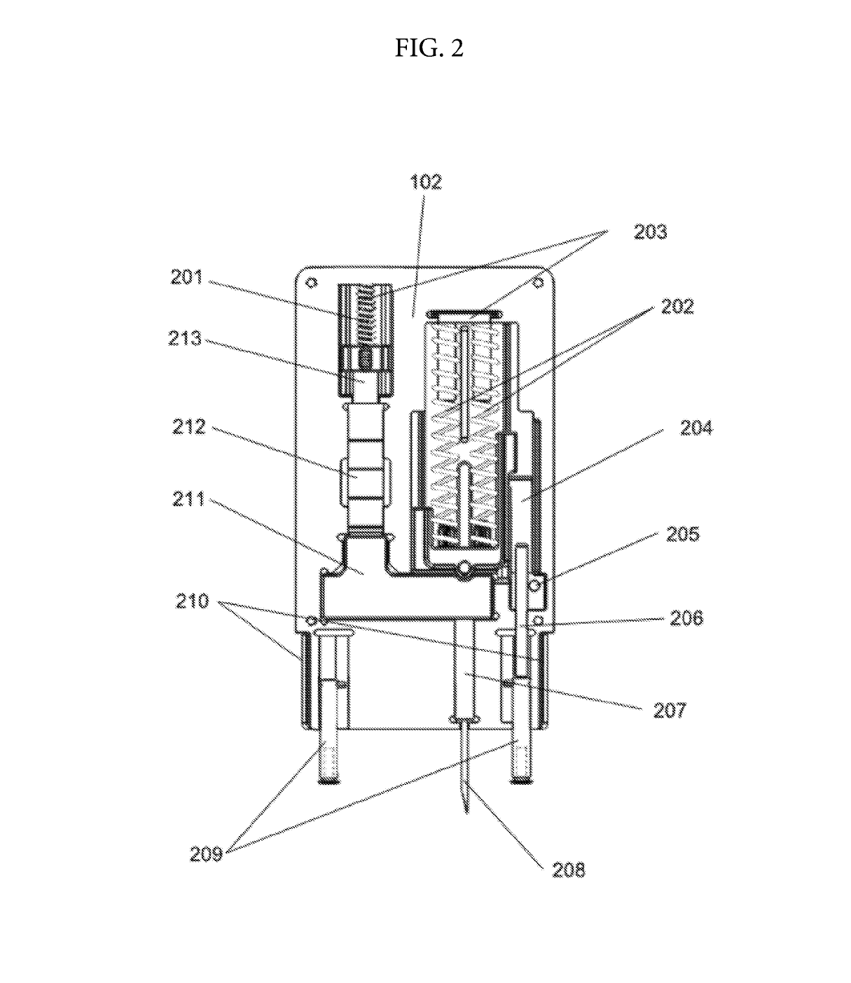

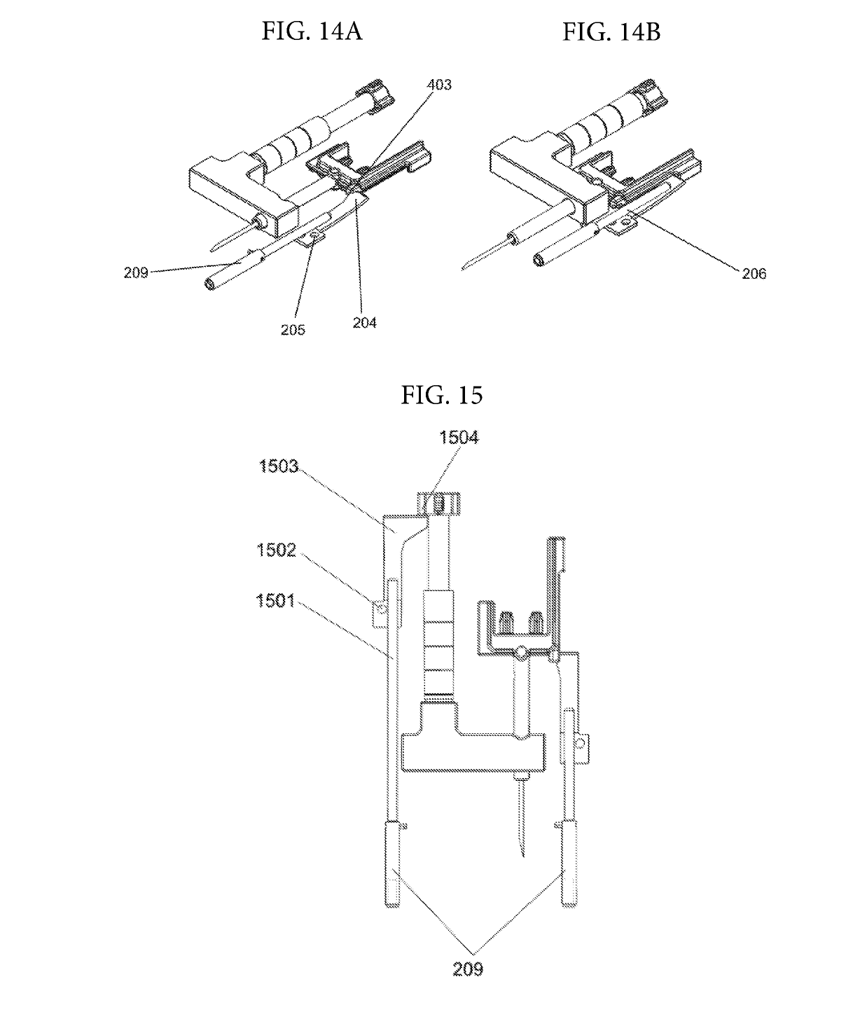

The triggering system includes an injection molded triggering mechanism component 106 that fits over the proximal end of the top and bottom housing components 101, 102. The triggering mechanism component 106 can be laterally guided from the proximal end of the auto-injector to the distal end of the auto-injector by a set of indented grooves or rails 210, which, as illustrated in FIG. 2, are extrusions off of the sides of the proximal end of the top and bottom housing components 101, 102. Attached to the triggering mechanism component 106 are two guide pins 209. The guide pines 209 are attached to the triggering mechanism component 106 through the use of two fasteners 108 that thread into tapped holes. The tapped holes are co-axial with the guide pins 209 on their proximal ends. These guide pins 209 extend from the proximal end of the auto-injector into grooves inside the housing. Also inside the housing are lateral roll-pins set into pilot holes on the guide pins 209 that restrict extension and compression of the trigger mechanism component 106. One of the guide pins 209 extends and pushes an activation/trigger pin 206 forward over an activation spring 204 to actuate the activation spring 204 and fire the auto-injector from its armed state, as illustrated in FIGS. 14A and 14B. In some embodiments, the activation spring 204 is a leaf spring.

The main functionality of the device is to accomplish two individual actions. The first is automatic actuation of a needle 208 from within the housing out through a small needle port hole 107 to a position outside of the housing and past the needle triggering component 106. The second action is expulsion of a dosage of medication through said needle 208 after the needle actuation process is completed.

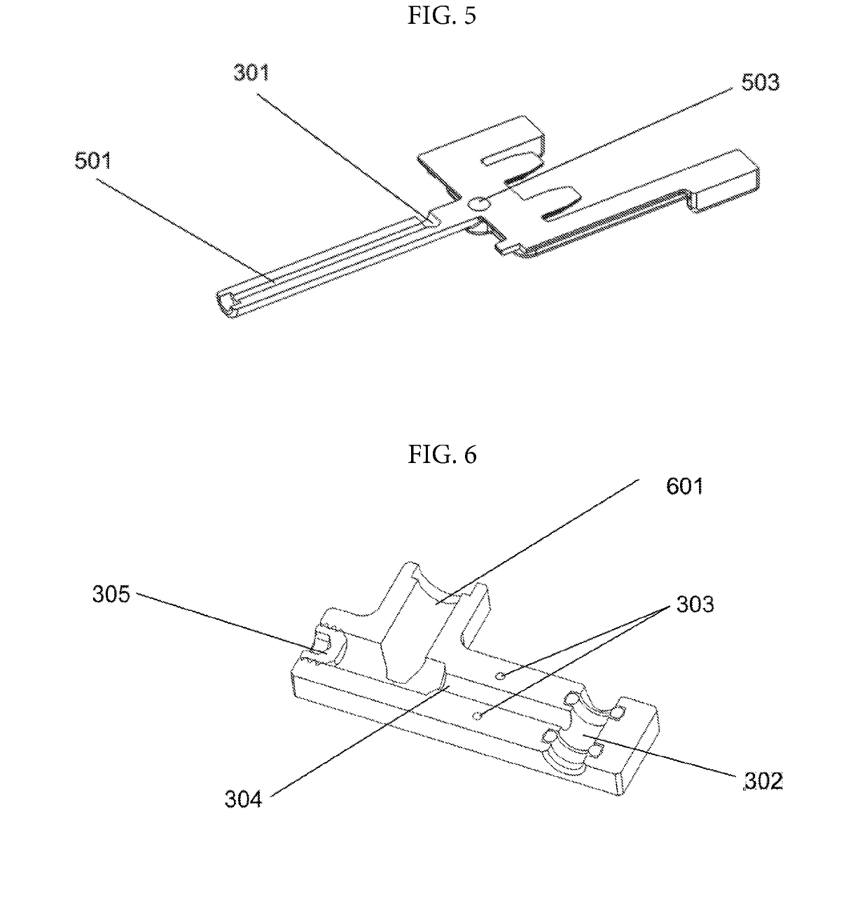

To complete these two actions, a unique arrangement of components are contained within the housing, as illustrated in FIG. 2. FIG. 2 is a top view of the internals of the auto-injector, with the top housing component 101, triggering mechanism component 106, and safety tab 105 removed and with the needle housing component 207 and the plunger housing component 213 in their post-activation states. In some embodiments, a needle 208 is over-molded, press-fit, or adhered into a needle housing component 207, illustrated in FIG. 5. The needle housing component 207 is uniquely shaped to hold the needle 208 within a needle channel 501. It can utilize the force from a first energy storage unit 202 to actuate the needle 208 out of the proximal end of the auto-injector. The first energy storage unit 202 can be in the form of a compression spring, a set of compression springs, an elastomer, or a compressed air canister such as a jet injector. When the needle 208 is actuated, its open aligns with the needle housing fluid communication port 301 and it is, therefore, connected to the fluid communication channel 304 and, subsequently, the medicament storage container. The needle housing component 207 may be machined or cast from a metallic material. Alternatively, the needle housing component 207 may be injection molded out of a thermoplastic material.

As briefly described above, activation of the auto-injector is achieved through the disengagement of an activation spring 204, such as a small leaf spring, from its locking position against a small catch 403 in the side of the needle housing component 207. The activation spring 204 is locked in place between the top and bottom housing components 101, 102 by a set of press-fit extrusion pins 205 on the bottom housing component 102. Disengagement of the activation spring 204 is achieved, as illustrated in FIGS. 14A and 14B, by removing the safety tab 105 and compressing the triggering mechanism component 106, which effectively slides the activation/trigger pin 206 over the activation spring 204. The of the activation spring 204, which is cantilevered up into the catch 403 when the needle housing component 207 is in its armed position, is then forced downward, away from the catch 403, allowing the needle housing component 207 to slide forward toward the proximal end of the device due to the force of the first energy storage unit 202. FIG. 4A is a perspective view of the main functional delivery mechanism of the injector and illustrates the orientation of the needle housing component 207, the plunger 307, and the plunger housing component 213 in the pre-activation state. FIG. 4B is a perspective view of the main functional delivery mechanism of the injector and illustrates the orientation of the needle housing component 207, the plunger 307, and the plunger housing component 213 in the post-activation state.

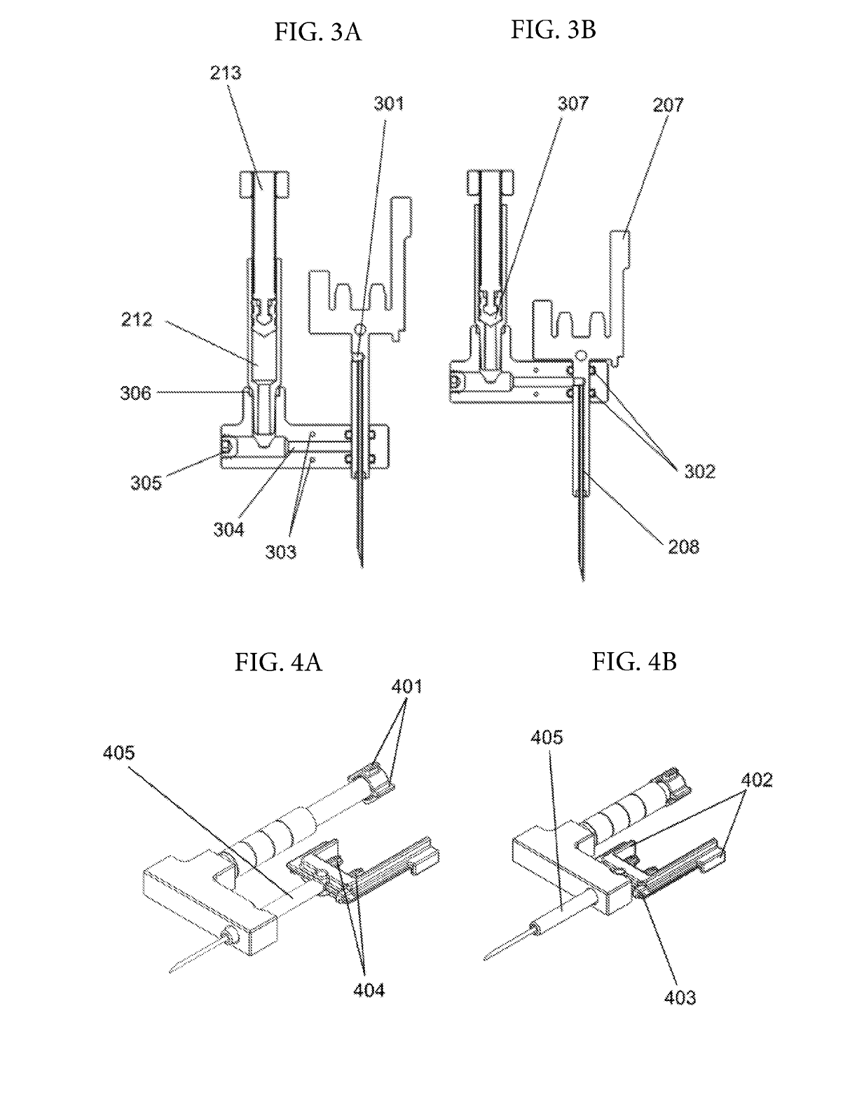

When triggered, the needle housing component 207 is guided forward by a set of alignment flanges 402 on either side of the first energy storage unit 202. In a preferred embodiment, the first energy storage unit 202 is a set of compression springs, which are press-fit onto a set of spring alignment pins 404. This allows the needle housing component 207 to be directed forward by the guiding rails in the top and bottom housing components 101, 102, which line up with the alignment flanges 402. The result is the actuation of the cylindrical barrel 405 on the needle housing component 207 through a pair of O-rings 302 housed within the fluid communication manifold 211, as illustrated in FIG. 6. The two O-rings 302 stack in line with the needle 208 and act to prevent fluid medication from inadvertently leaking out of the fluid communication manifold 211. More specifically, actuation of the cylindrical barrel 405, which holds the needle 208, results in alignment of the needle housing fluid communication port 301 with the fluid communication channel 304 of the fluid communication manifold 211, as illustrated in FIG. 3B. FIG. 3A is a cross-sectional view of the main delivery mechanisms of the auto-injector with the needle housing component 207 and the plunger housing component 213 in their pre-activation states. FIG. 3B is a cross-sectional view of the main delivery mechanisms of the auto-injector with the needle housing component 207 and the plunger housing component 213 in their post-activation states.

In one embodiment of the auto-injector, a resetting pin hole 503 exists in the needle housing component 207 that, in conjunction with a groove in the top and bottom housing components 101, 102, allows for the insertion of a small pin through the housing. This allows a user to reset the needle housing component 207, activation spring 204, and first energy storage unit 202 to their armed state after use and aids a user that is training to use the auto-injector. In some embodiments, the needle port hole 107 may contain a rubber seal capable of being punctured that provides a boundary to protect the needle 208 from dirt, dust, and microbial particles.

As illustrated in FIG. 15, one embodiment of this design may include a second triggering system that uses a leaf spring 1503 to control the release of medication from the medicament storage container. The leaf spring 1503 can release a locked plunger mechanism from its armed state. More specifically, one of the guide pins 209 can push a second trigger activation pin 1501 forward over the leaf spring 1503 to actuate the leaf spring 1503 and release the catch 1504. The catch 1504 can then provide pressure to the syringe system, which causes the medication to flow out of the syringe system. The leaf spring 1503 is attached to the housing using a set of press-fit extrusion pins 1502, which are similar to the press-fit extrusion pins 205 in the first mechanism. The second triggering system can, in some embodiments, be activated simultaneously with the first triggering system that controls actuation of the needle housing component 207. Activation of both triggering systems facilitates full activation of the auto-injector.

The fluid communication manifold 211, as illustrated in FIG. 6, works to move fluid from a medicament storage container to the needle housing fluid communication port 301 of the needle housing component 207. It does this through the use of positive pressure from a second energy storage unit 201, 702, which is responsible for pushing the medication through the fluid communication channel 304 after the needle housing component 207 is activated.

The fluid connection manifold 211 is precisely positioned within the housing by the use of a set of guidance extrusions on the bottom housing component 102. The guidance extrusions align with the positioning holes 303 in the fluid communication manifold 211. The fluid communication manifold 211 can be constructed of a cast or machined metal. Alternatively, it can be an injection molded plastic or a plastic/glass combination such as glass-filled nylon. As this particular embodiment is designed for machining processes, the fluid communication manifold 211 contains a thread-locked set screw 305, which closes the port that allows for the manufacturing of this part. In the case of injection molding, a similar component would need to exist to allow the molding tool to exit. At the connection point 601 of the medicament storage container to the fluid communication manifold 211, there can be a small O-ring seal 306 to ensure there is no fluid leakage between the syringe 212 and the fluid communication manifold 211.

In one embodiment, the medicament storage container is comprised of a syringe 212 and plunger 307, as illustrated in FIGS. 3A and 3B. The medication in the syringe 212 can be under constant pressure from the second energy storage unit 201, which, when released by the second triggering system, is guided by a guide pin 203 in the housing and a metal or plastic plunger housing component 213. The plunger housing component 213 can hold the plunger 307 within the syringe 212 and can align in the housing through the use of plunger alignment flanges 401. The plunger alignment flanges 401 can assist in guiding the plunger housing component 213 along its proper path within the housing as the auto-injector is activated and fluid medication is expelled.

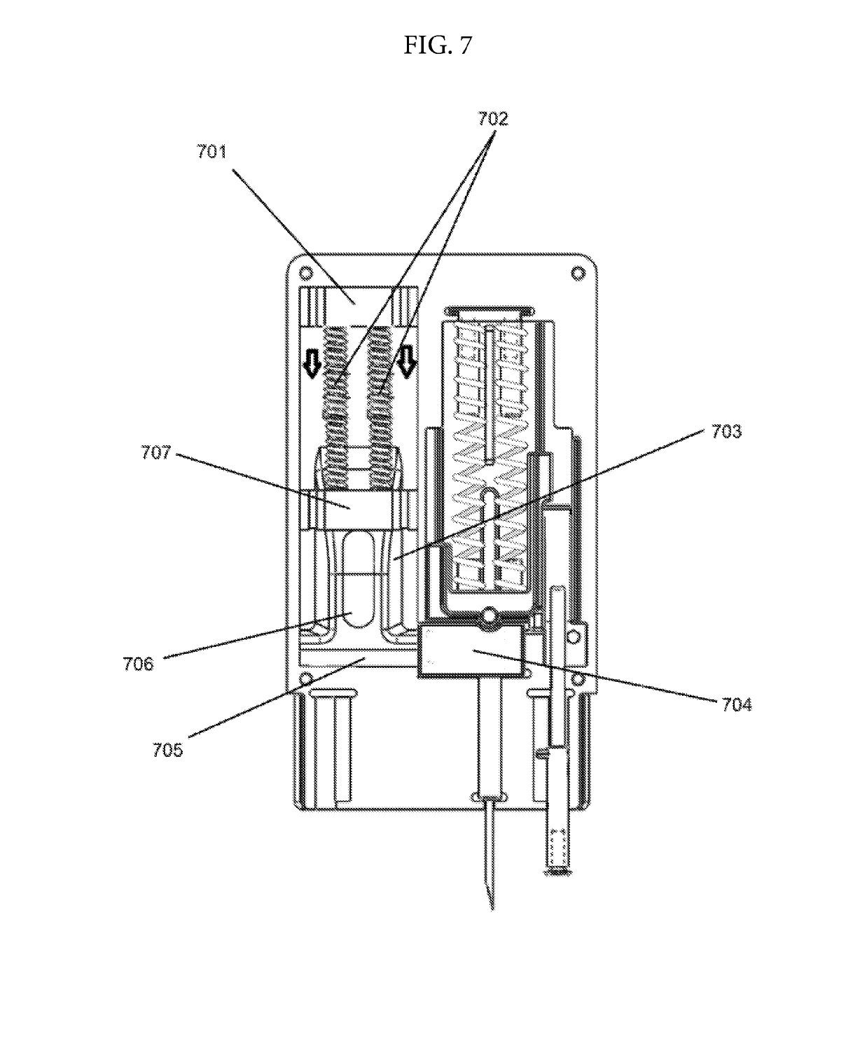

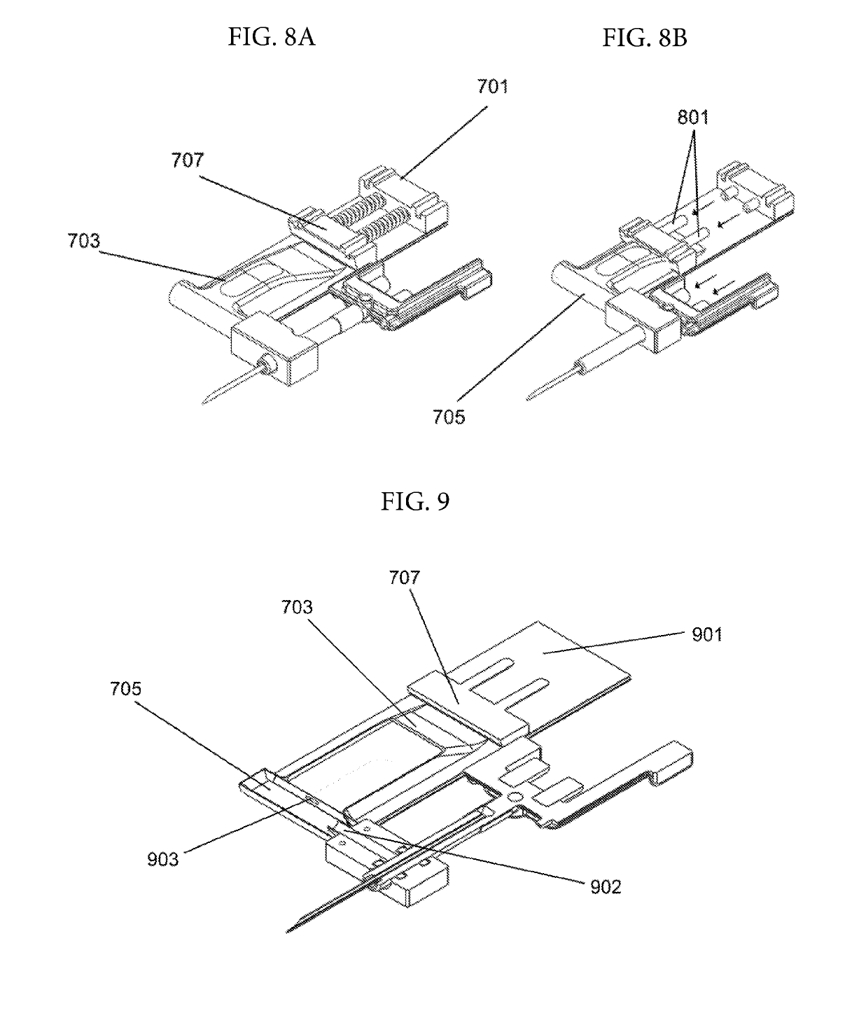

In another embodiment of the auto-injector, the medicament storage container is a medical-grade, collapsible plastic pouch 703. FIG. 7 illustrates a top view of the internals of the auto-injector, wherein the plunger housing component 213 is replaced with a collapsible pouch 703, the needle housing component 207 is in a post-activation state, and the pushbar 707 is partially through its firing action. FIG. 8A illustrates an embodiment of the auto-injector that uses a collapsible pouch 703 and pushbar 707, wherein the auto-injector is in its pre-activation state. FIG. 8B illustrates an embodiment of the auto-injector that uses a collapsible pouch 703 and pushbar 707, wherein the auto-injector is in its post-activation state.

As illustrated in FIG. 9, the collapsible pouch 703 can be thermally bonded to a frame 901 that serves to provide both structure and specific geometries to the size and shape of the collapsible pouch 703. The collapsible pouch 703 and pushbar 707 serve a similar purpose to that of the syringe 212 and plunger 307. More specifically, they can store medication under constant pressure from a second energy storage unit 702. The second energy storage unit 702 can be in the form of a compression spring, a set of compression springs, compressed air, or an elastomer. In the case of a pair of compression springs, the springs are aligned using guide pins 801 that are located on the housing guide insert 701 and the compression sliding pushbar 707, as illustrated in FIGS. 8A and 8B. To provide pressure to the collapsible pouch 703, the pushbar 707 can move over the collapsible pouch 703 and push the top and bottom of the collapsible pouch 703 together. At least a portion of the collapsible pouch 703 can be made of clear or translucent material 706 in order to allow the user to see through the viewing ports 104 in the housing. This enables a user to view the status of the medication and determine if the medication is discolored or if it contains unwanted precipitates.

The collapsible pouch embodiment of the auto-injector may require a modified fluid communication manifold 704, as illustrated in FIG. 9, that facilitates fluid flow from the collapsible pouch 703. For example, fluid flows from the collapsible pouch 703, through the fluid regulation port 903, into the fluid communication channel 705 of the pouch frame 901, into the channel 902 of the modified fluid communication manifold 704, and out through the needle 208 when in its activated position.

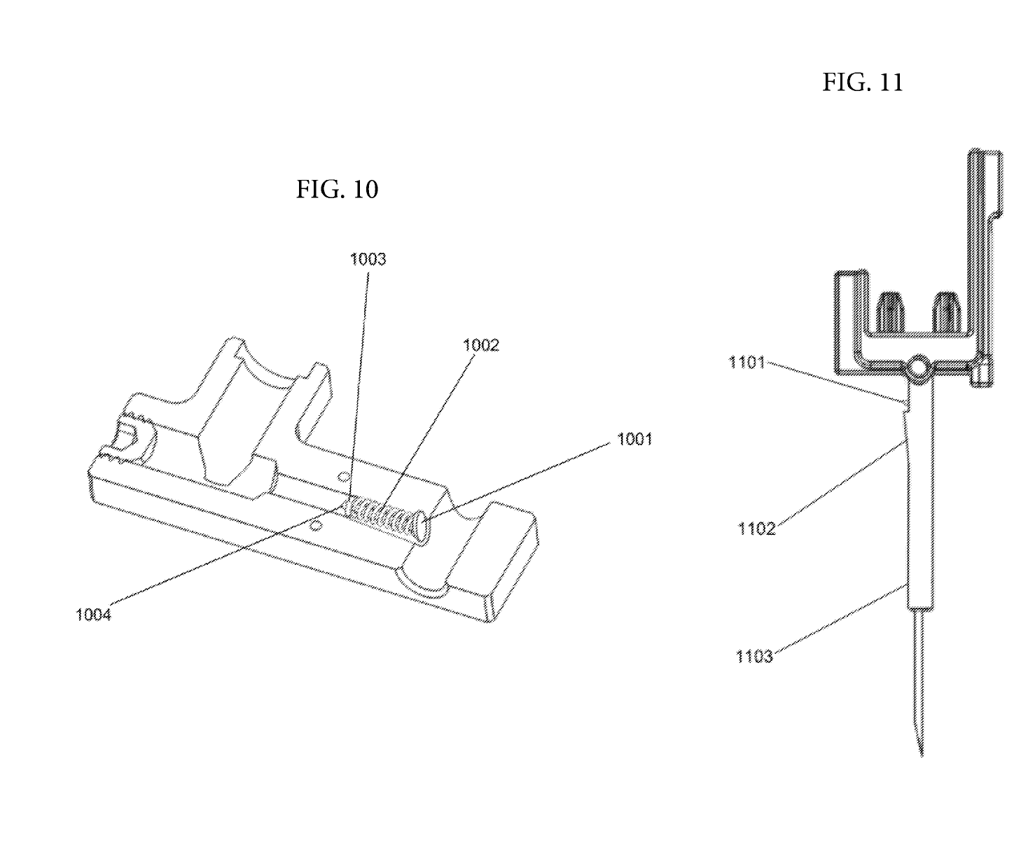

In another embodiment of the auto-injector, the seal created by the set of geometrical O-rings 302 is replaced by a frangible seal 1001. As illustrated in FIGS. 10 and 11, as the needle housing component 207 is actuated to its fired position by the first energy storage unit 202, the frangible seal 1001 travels across the length of the flat surface on the side 1103 of the cylindrical barrel 405. A compression spring 1002 can keep pressure on the frangible seal 1001, which is anchored through the use of a tube 1004 that runs inside of, and co-axial with, the compression spring 1002 and is connected to the fluid communication channel 304. The compression spring 1002 can be locked in place between an o-ring seal 1003 and the frangible seal 1001. The o-ring seal 1003 can also keep fluid medication from entering the compartment in which the compression spring 1002 and tube 1004 are held. Once the frangible seal 1001 reaches the ramp 1102 on the side of the cylindrical barrel 405, the compression spring 1002 will compress toward the o-ring seal 1003, and the frangible seal 1001 will give way as the ramp 1102 passes. Once the ramp 1102 has passed, the frangible seal 1001 will be forced quickly forward by the compression spring 1002 and onto a puncturing metal needle point 1101 on the cylindrical barrel 405. The puncturing metal needle point 1101 can break the frangible seal 1001 and allow the fluid medication to flow from the fluid communication channel 304, through the tube 1004, past the punctured frangible seal 1001, and into the needle 208, which is now in its fired state with its needle housing fluid communication port 301 aligned with the fluid communication channel 304.

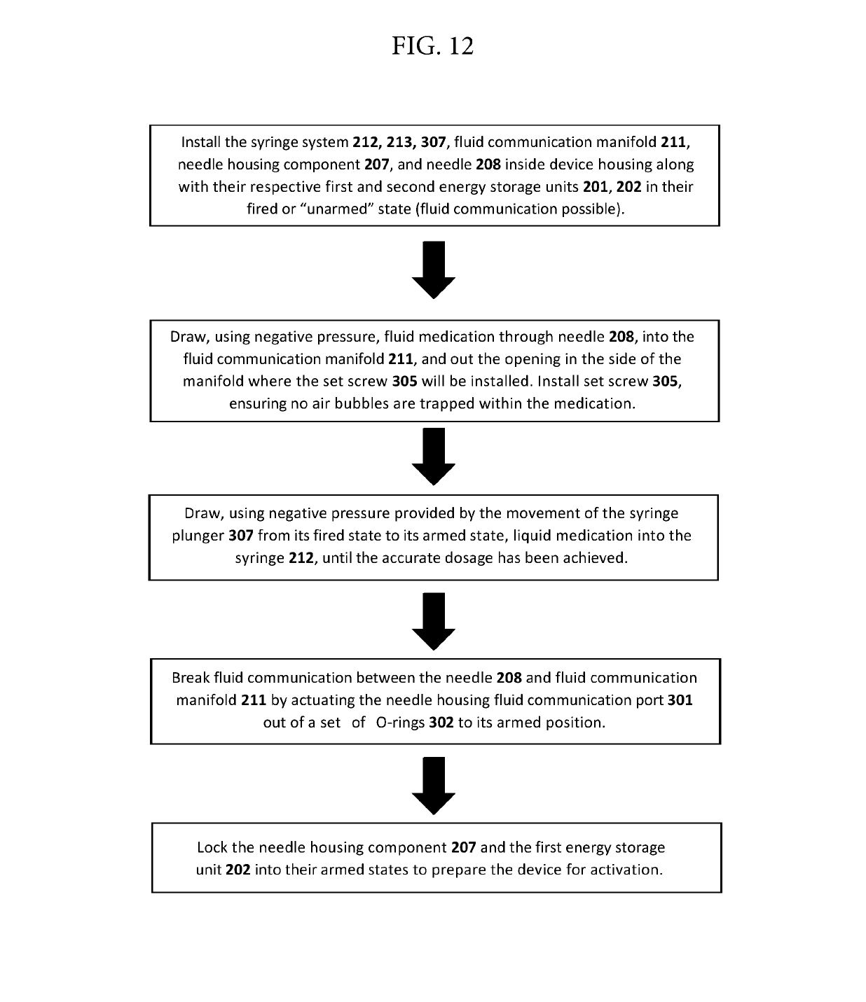

The filling process for the disclosed auto-injector is a unique process, and two embodiments of the filling process are fully described herein. A first process, illustrated in FIG. 12, is comprised of the following steps. First, install a syringe system 212, 213, 307, fluid communication manifold 211, needle housing component 207, and needle 208 inside a device housing along with the first and second energy storage units 201, 202 in post-activation states. Second, draw, using negative pressure, fluid medication through the needle 208 into the fluid communication manifold 211 and out the opening in the side of the fluid communication manifold 211 where a set screw 305 is then installed ensuring no air bubbles are trapped within the fluid medication. Third, draw, using negative pressure provided by the movement of the plunger 307 from its post-activation state to its pre-activation state, fluid medication into the syringe 212 until the accurate dosage has been achieved. Fourth, break fluid communication between the needle 208 and the fluid communication manifold 211 by actuating the needle housing fluid communication port 301 out of a set of O-rings 302 into its pre-activation state. Fifth, lock the needle housing component 207 and the first energy storage unit 202 into their pre-activation states to prepare the auto-injector for activation.

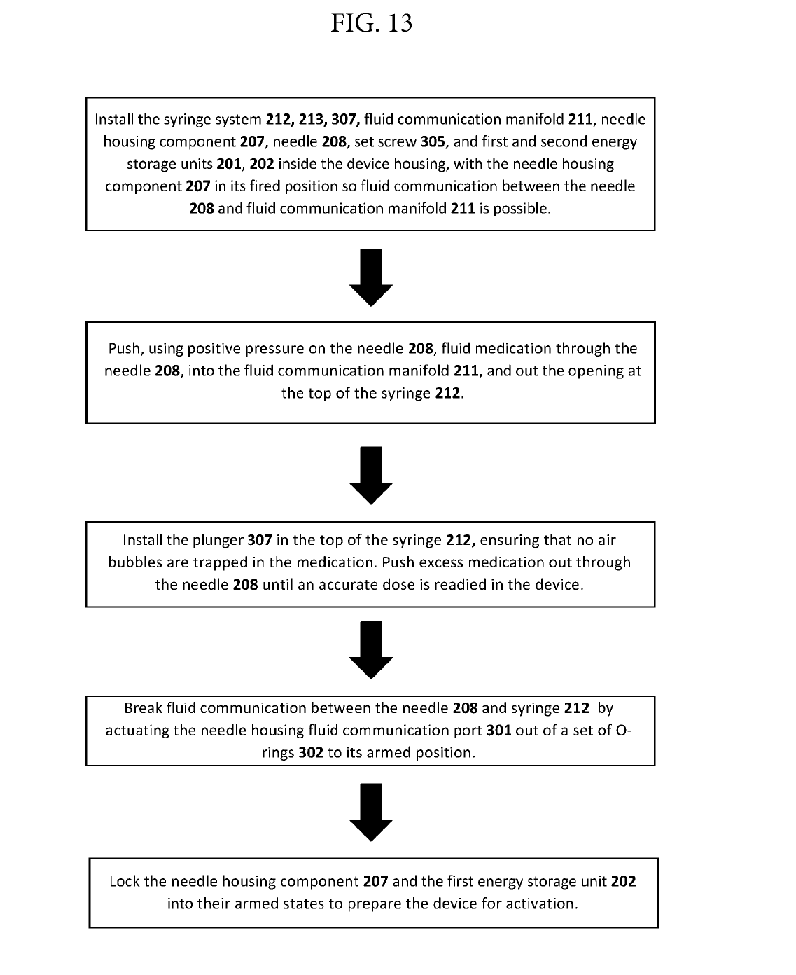

A second process of filling the auto-injector, illustrated in FIG. 13, is comprised of the following steps. First, install a syringe system 212, 213, 307, fluid communication manifold 211, needle housing component 207, needle 208, set screw 305 and first and second energy storage units 201, 202 inside the device housing, with the needle housing component 207 in its post-activation state so that fluid communication between the needle 208 and the fluid communication manifold 211 is possible. Second, push, using positive pressure on the needle 208, fluid medication through the needle 208, into the fluid communication manifold 211, and out of the opening at the top of the syringe 212, which is where the plunger 307 is to be installed. Third, install the plunger 307 in the top of the syringe 212, ensuring that no air bubbles are trapped in the medication, and push excess medication out through the needle 208 until the accurate dose is readied in the auto-injector. Fourth, break fluid communication between the needle 208 and the syringe 212 by actuating the needle housing fluid communication port 301 out of a set of O-rings 302 to its pre-activation state. Fifth, lock the needle housing component 207 and the first energy storage unit 202 in their pre-activation states to prepare the auto-injector for activation.

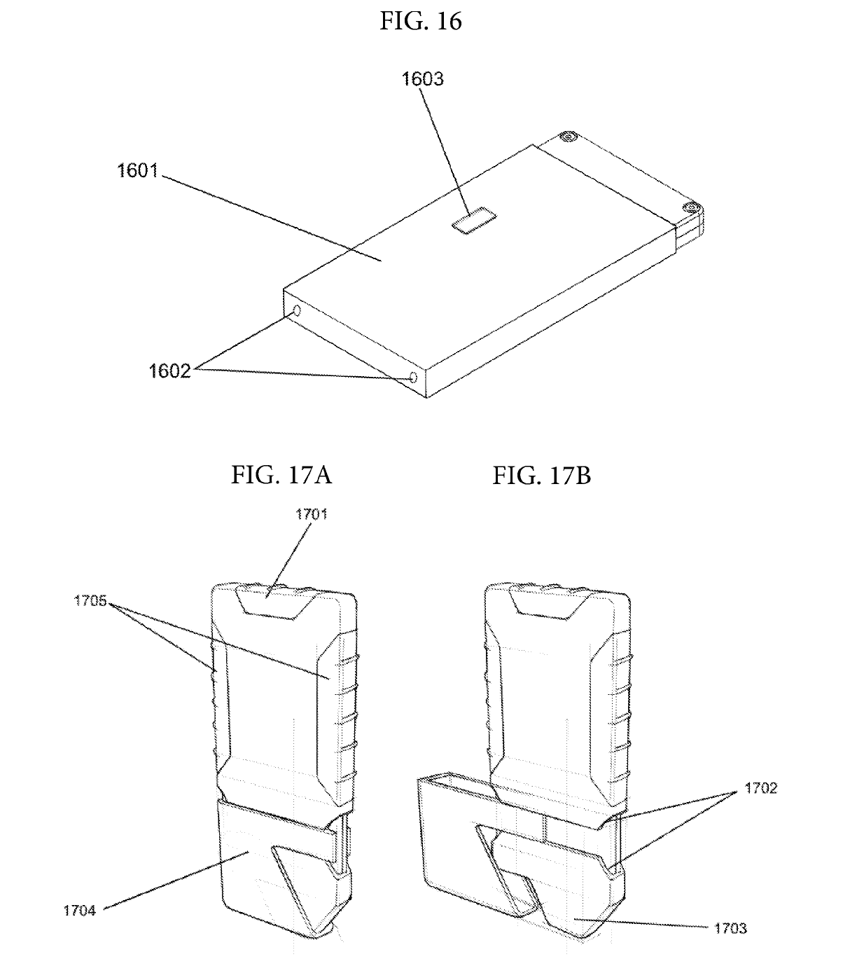

In addition to the embodiments described above, additional possible embodiments for the form factor of the auto-injector exist that include various cases, safety tabs, and instructive shapes, and these embodiments are illustrated in FIGS. 16 through 24, the details of which are described below.

As described above, the auto-injector may be stored in an external carrying case 1601, as illustrated in FIG. 16. The external case 1601 may contain an acrylic, Plexiglas, glass, or otherwise clear window 1603. This window 1603 can allow the user to see through the external case 1601, the viewing port 104 aligned underneath the window 1603, and the syringe 212 in order to check for discoloration of medication or unwanted precipitants. The external case 1601 can also contain air release ports 1602 on one of the external case 1601 to allow for air to enter the external case 1601 as the auto-injector is pulled out from the opposite end. The external case 1601 may also include a thermal barrier such as an aluminum backed adhesive or other thermally protective coating on the inside or outside of the external case 1601 to protect the auto-injector and medication from adverse heat elements.

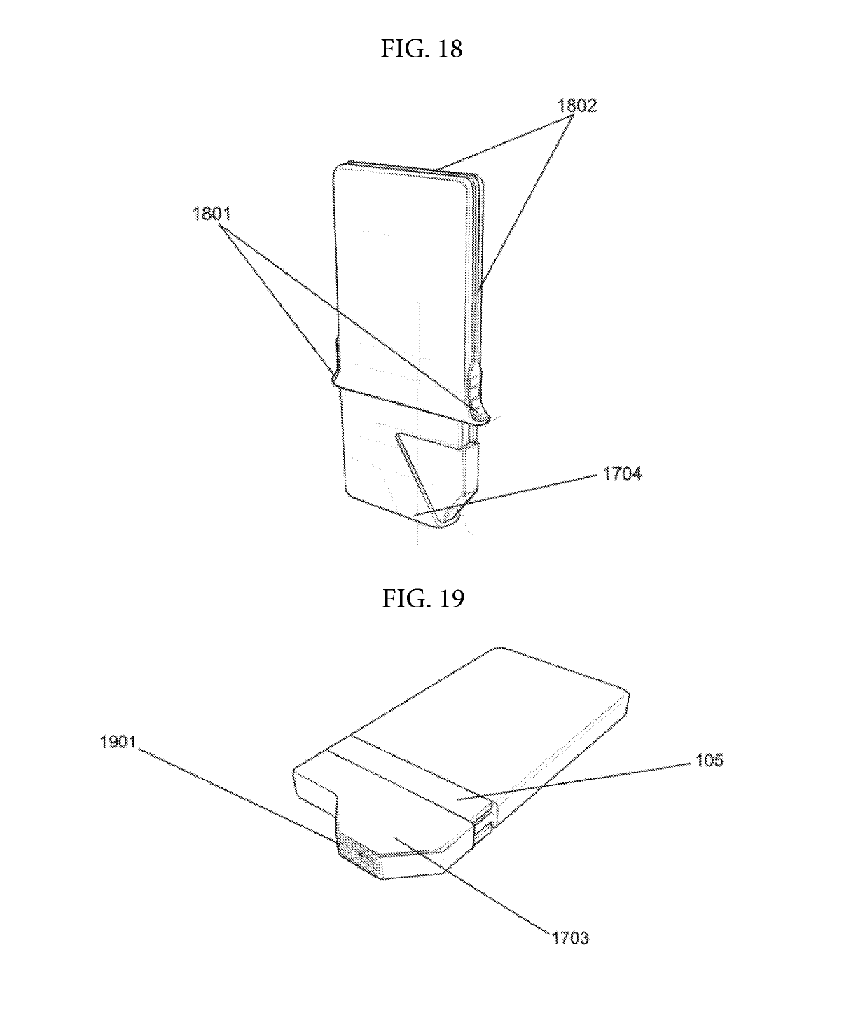

In one embodiment, the auto-injector housing may have rubber, silicone, hard plastic, or other, high-friction based material 1701, 1705 on its sides, as illustrated in FIG. 17A, to allow the user to more easily hold the auto-injector during the action of injection. In some embodiments, as illustrated in FIG. 18, additional gripping abilities may exist through the inclusion of grip flanges 1801 on the side of the auto-injector as well as a long, thin grip strip 1802 on the edges of the auto-injector.

Some embodiments of the disclosed invention may also include anti-pinch cut-outs 1702 in the top and bottom housing components 101, 102 and in the triggering mechanism component 1703, as illustrated in FIG. 17B, to allow the user to grip the sides of the device without risk of pinching skin between the moving components during injection. Further, some embodiments can contain a unique safety cover 1704, illustrated in FIG. 17A, that operates as a protective safety mechanism and cover. More specifically, it can encompass the role of the safety tab 105 described above, and it can cover the needle port hole 107, thereby further protecting the needle 208 from dirt, dust, and microbial particles. When a user is ready to use the auto-injector, the safety cover 1704 can be removed, as illustrated in FIG. 17B, which simultaneously exposes the needle port hole 107 and enables the triggering mechanism component 1703 to move from the proximal end of the auto-injector to the distal end of the auto-injector.

In a further embodiment, a safety tab 105 may be included as well as gripping cleats 1901 on the triggering mechanism component 1703, as illustrated in FIG. 19. The gripping cleats 1901 may be made of a rubber or hard plastic material and can keep the auto-injector from slipping on the user's injection site.

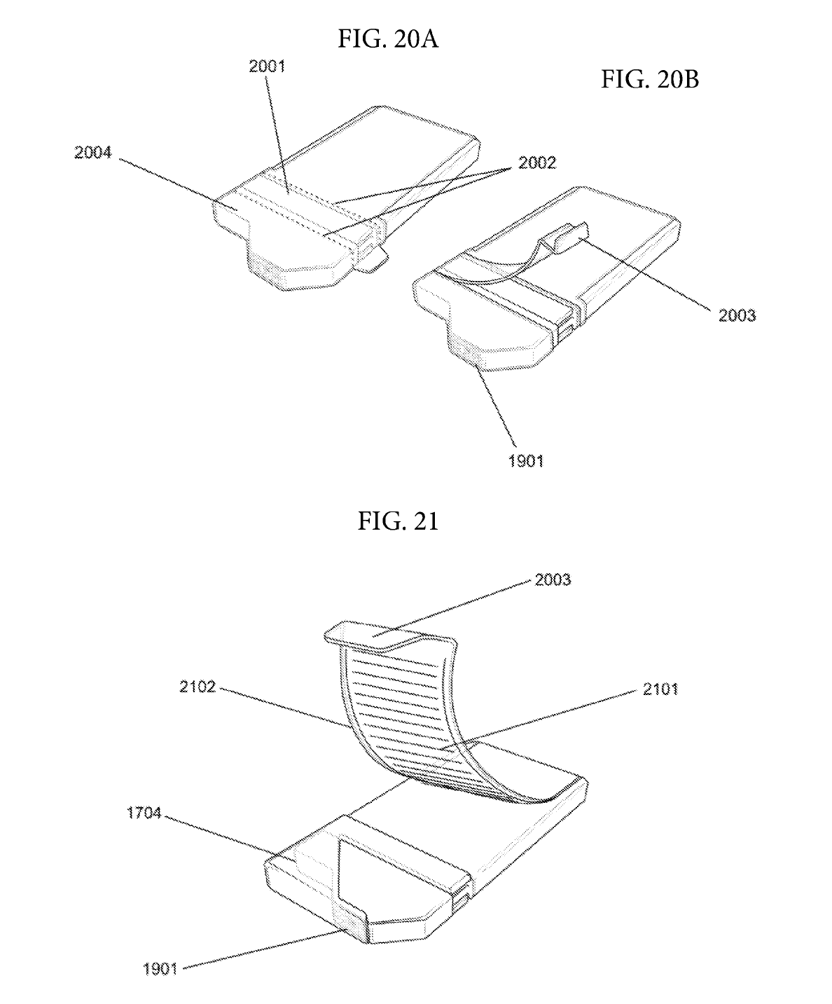

In some embodiments, the triggering mechanism component 2004 may be covered by a paper or plastic seal 2001, as illustrated in FIG. 20A. The paper or plastic seal 2001 may protect the triggering mechanism component 1703 and gripping cleats 1901 from dirt, debris, and microbial particles. To remove the paper or plastic seal 2001 from the triggering mechanism component 2004, the paper or plastic seal 2001 may be torn via the pulling of a tab 2003, as illustrated in FIG. 20B, and the tearing of perforated tear lines 2002 so that the section of the paper or plastic seal 2001 covering the safety tab 105 is removed, thereby allowing the auto-injector to be further activated.

As illustrated in FIG. 21, one embodiment of the disclosed invention may include a paper or plastic flap 2102 that is hinged on the top of the auto-injector and adhered in place with the use of a tab 2003 on the bottom edge of the device. This flap 2102 may operate as a sealing mechanism over the safety cover 1704. On the inside of the flap 2102, there may be labeling 2101 that assists the user in the activation process.

Because of the compact design of the auto-injector, it may be more easily transported than current auto-injectors that exist in the market. For example, the disclosed auto-injector may be attached to a belt via a belt clip, it may be adhered to a phone or phone case, or it may be kept in a wallet. These are all locations that are more accessible for a user than a bag.

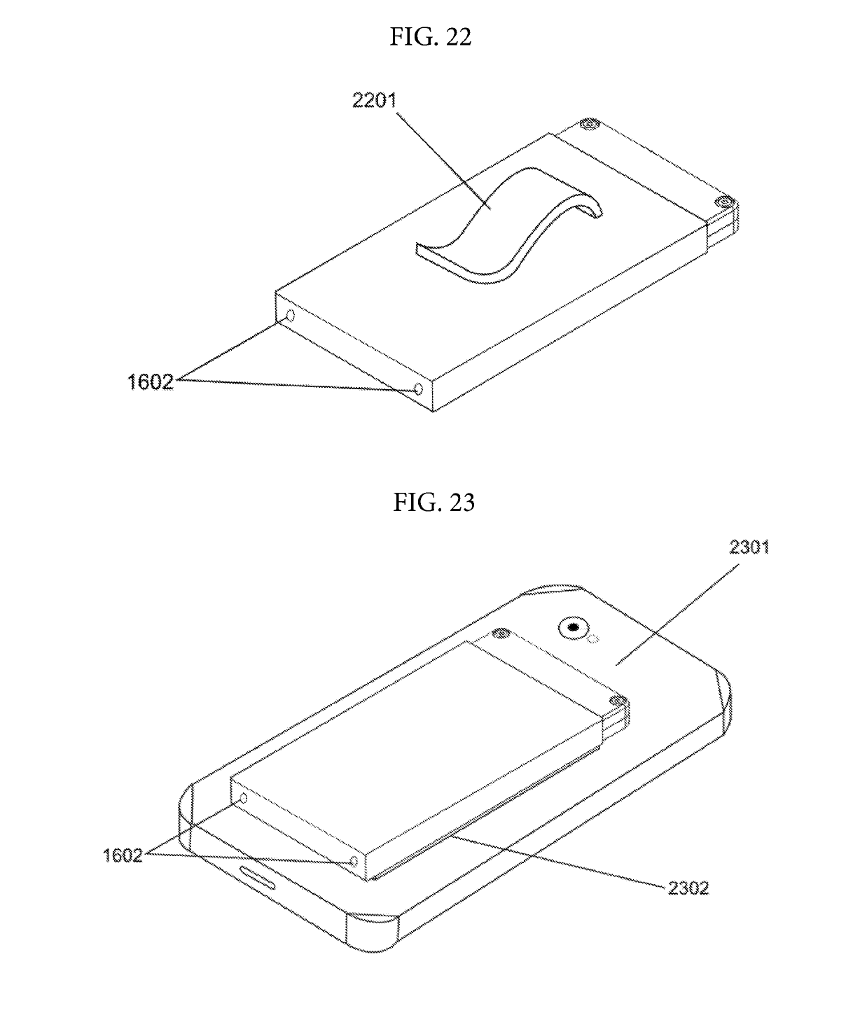

More specifically, in some embodiments, the external case 1601 may have a belt clip 2201 or a key-chain loop built into or attached to it, as illustrated in FIG. 22. The belt clip 2201 may allow for easier carrying of the compartmentalized auto-injection system. Instead of a belt clip 2201, the external case 1601 may have an adhesive connection 2302 to join the external case 1601 or any usability embodiment of the design to the rear face of a standard cell phone or other electronic device 2301 to allow for easier carrying of the compartmentalized auto-injection system, as illustrated in FIG. 23. The adhesive connection 2302 may also provide a thermal barrier between the external case 1601 and the electronic device 2301 to protect the medication from any adverse heat. Another similar embodiment may be a case for the electronic device 2301 that has an integrated slot for holding the auto-injector itself.

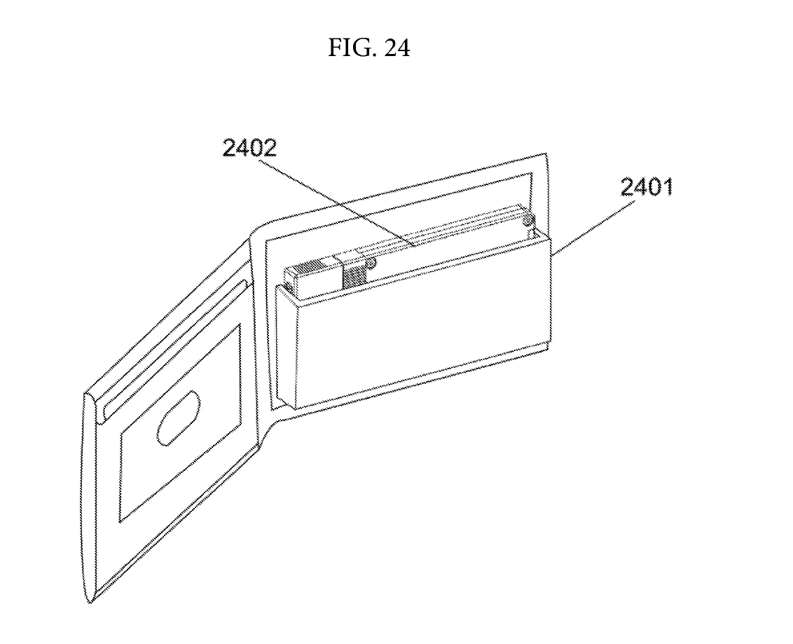

FIG. 24 depicts another embodiment of the auto-injector, in which there exists a protective case 2401 held within a wallet insert, custom built wallet, or external protective case fashioned to hold items such as credit cards, cash, and identification cards. The protective case 2401 can hold any embodiment of the disclosed design 2402.

The various embodiments described above are provided by way of illustration only and should not be construed to limit the claims attached hereto. Those skilled in the art will readily recognize various modifications and changes that may be made without following the example embodiments and applications illustrated and described herein and without departing from the true spirit and scope of the following claims.

1. A compartmentalized auto-injection system comprising:
a housing;
a moveable triggering mechanism component attached to a proximal end of the housing for activating the compartmentalized auto-injection system;
a removable safety tab that is attached to the housing and located near a distal end of the moveable triggering mechanism component;
a needle port hole located on a proximal end of the moveable triggering mechanism component;
a needle;
a needle housing component that houses the needle and has a fluid communication port;
a medicament storage container that stores a fluid medication;
a fluid communication manifold comprised of a connection point to the medicament storage container, a fluid communication channel, and a seal that prevents the fluid medication from coming into fluid communication with the needle;
a first energy storage unit capable of breaking the seal, driving the needle through the needle port hole, and aligning the fluid communication port with the needle and the fluid communication channel;
a second energy storage unit capable of driving the fluid medication out of the medicament storage container, through the fluid communication channel, through the fluid communication port, and into, and out of, the needle;
a moveable guide pin attached on a first to the moveable triggering mechanism component and on a second to a moveable activation pin; and
an activation spring located near the moveable activation pin, wherein the activation spring controls activation of the first energy storage unit and the moveable activation pin is capable of activating the activation spring;
wherein the housing contains the needle, the needle housing component, the medicament storage container, the fluid medication, the fluid communication manifold, the first energy storage member, the second energy storage member, the moveable guide pin, the moveable activation pin, and the activation spring.

2. The compartmentalized auto-injection system of claim 1, wherein the medicament storage container is comprised of a syringe that stores the fluid medication and a plunger.

3. The compartmentalized auto-injection system of claim 1, wherein the medicament storage container is comprised of a collapsible pouch that stores the fluid medication and a pushbar.

4. The compartmentalized auto-injection system of claim 1, wherein the seal is a geometrical seal comprised of a pair of o-rings stacked in line with the needle and the needle housing component.

5. The compartmentalized auto-injection system of claim 1, wherein:
the seal is a frangible seal connected to a compression spring and a first of a tube;
a second of the tube is open to the fluid communication channel;
an o-ring seal holds the tube in place and prevents the fluid medication from coming into fluid communication with the compression spring;
the needle housing component is further comprised of a cylindrical barrel that contains the needle and is comprised of a ramp and a puncturing needle point; and
the ramp and the puncturing needle point are oriented in the direction of the frangible seal.

6. The compartmentalized auto-injection system of claim 1, further comprising an external case for containing the housing, the moveable triggering mechanism component, and the removable safety tab.

7. The compartmentalized auto-injection system of claim 6, wherein the external case has an adhesive connection for attaching the external case to an electronic device.

8. The compartmentalized auto-injection system of claim 1, wherein a portion of the housing is a clear viewing port and a portion of the medicament storage container is clear.

9. The compartmentalized auto-injection system of claim 1, wherein:
the activation spring is a leaf spring;
the activation spring is locked in place against a catch by a set of press-fit extrusions; and
the catch is configured to prevent activation of the first energy storage unit.

10. The compartmentalized auto-injection system of claim 1, further comprising a second moveable guide pin attached on a first to the moveable triggering mechanism component and on a second to a second moveable activation pin.

11. The compartmentalized auto-injection system of claim 10, further comprising a second activation spring located near the second moveable activation pin, wherein the second activation spring controls activation of the second energy storage unit and the second moveable activation pin is capable of activating the second activation spring.

12. The compartmentalized auto-injection system of claim 11, wherein:
the second activation spring is a leaf spring;
the second activation spring is locked in place against a second catch by a second set of press-fit extrusions; and
the second catch is configured to prevent activation of the second energy storage unit.

13. The compartmentalized auto-injection system of claim 1, wherein the first energy storage unit is comprised of a pair of compression springs.

14. The compartmentalized auto-injection system of claim 1, wherein the second energy storage unit is comprised of a compression spring.

15. The compartmentalized auto-injection system of claim 1, wherein the proximal end of the moveable triggering mechanism component is uniformly flat.

16. The compartmentalized auto-injection system of claim 1, wherein a portion of the proximal end of the moveable triggering mechanism component comes to a flat point and the needle port hole is located on the flat point.

17. The compartmentalized auto-injection system of claim 16, wherein the removable safety tab wraps around the proximal end of the moveable triggering mechanism and covers the needle port hole.

18. The compartmentalized auto-injection system of claim 1, wherein the housing is comprised of a top housing component attached to a bottom housing component.

19. An auto-injector comprising:
a housing;
a needle housing component, moveable with respect to the housing, including a fluid communication port;
a medicament storage container, fixed with respect to the housing, the medicament storage container including a needle component channel configured to receive the needle housing component, and a seal; and
an energy storage member that, when activated, is configured to move the needle housing component in the needle component channel from a first position, where the seal of the medicament storage container is intact, to a second position, where the seal is broken, fluidly connecting the medicament storage container with the fluid communication port.

20. The auto-injector of claim 19, wherein the medicament storage container includes a fluid communication manifold, fixed with respect to the housing, the fluid communication manifold including a fluid communication channel configured to receive a fluid medication, the needle component channel configured to receive the needle housing component, and the seal,
wherein the seal includes first and second O-rings in the needle component channel on opposite sides of the fluid communication channel, and
wherein the energy storage member, when activated, is configured to move the needle housing component in the needle component channel from the first position, wherein the fluid communication port is outside of the first and second O-rings, to the second position, wherein the fluid communication port aligns with the fluid communication channel between the first and second O-rings, breaking the seal, and fluidly connecting the fluid communication channel with the fluid communication port.

21. The auto-injector of claim 20, wherein the needle housing component includes a needle having a lumen, wherein the lumen is in fluid communication with the fluid communication port when the fluid communication port aligns with the fluid communication channel, the auto-injector further comprising:
a triggering mechanism component coupled to a distal end of the housing, the triggering mechanism component including:
a needle port on a distal end of the triggering mechanism component, the needle port configured to receive the distal end of the needle therethrough when the triggering mechanism is activated.

22. The auto-injector of claim 21, further comprising:
a second energy storage member activated by the triggering mechanism component together with the energy storage member, the second energy storage member configured to motivate fluid medicament to flow from the medicament storage container through the fluid communication channel, through the communication port, and to the needle; and
a plunger disposed within the medicament storage container and connected to the second energy storage member, the plunger configured to use energy from the second energy storage member to motivate fluid medicament to flow from the medicament storage container when the trigger is in the second position.

23. The auto-injector of claim 19, wherein the housing has a length, width, and depth, wherein the length is greater than the width, and the width is greater than the depth,
wherein the needle housing component includes a needle having a lumen in fluid communication with the fluid communication port,
wherein, when the needle housing component is in the first position, the distal end of the needle is contained within the housing, and
wherein, when the needle housing component is in the second position, the distal end of the needle is outside of the housing.

24. The auto-injector of claim 23, wherein the fluid communication port is substantially normal to the length of the housing, and
wherein the needle housing component is movable from the first position to the second position along an axis substantially normal to the fluid communication port.

25. A method comprising:
moving, using an energy storage member, a needle housing component of an auto-injector in a needle component channel of a medicament storage container from a first position, wherein a seal of the medicament storage container is intact, to a second position, breaking the seal of the medicament storage container and fluidly connecting an interior of the medicament storage container with a fluid communication port of the needle housing component,
wherein the medicament storage container is fixed with respect to a housing of the auto-injector.

26. The method of claim 25, wherein, in the first position, the fluid communication port is outside of first and second O-rings in the needle component channel on opposite sides of a fluid communication channel configured to receive fluid medication from the medicament storage container, and
wherein, in the second position, the fluid communication port aligns with the fluid communication channel between the first and second O-rings, breaking a seal created by the first and second O-rings and fluidly connecting the fluid communication channel with the fluid communication port.

27. The method of claim 26, further comprising:
triggering, using a triggering mechanism component coupled to a distal end of a housing, a first energy storage mechanism to move the needle housing component from the first position to the second position,
wherein the needle housing component includes a needle having a lumen in fluid communication with the fluid communication port when the fluid communication port aligns with the fluid communication channel.

28. The method of claim 27, further comprising:
triggering, using the triggering mechanism component, a second energy storage member to move a plunger disposed within the medicament storage container to motivate fluid medicament to flow from the medicament storage container through the fluid communication channel, through the communication port, and to the needle.

29. The method of claim 25, wherein the housing has a length, width, and depth, wherein the length is greater than the width, and the width is greater than the depth,
wherein the needle housing component includes a needle having a lumen in fluid communication with the fluid communication port,
wherein, when the needle housing component is in the first position, the distal end of the needle is contained within the housing, and
wherein, when the needle housing component is in the second position, the distal end of the needle is outside of the housing.

30. The method of claim 29, wherein the fluid communication port is substantially normal to the length of the housing, and
wherein the needle housing component is movable from the first position to the second position along an axis substantially normal to the fluid communication port.
